(19) United States
(12) Patent Application Publication
(10) Pub. No.: US 2004/0259934 A1
(43) Pub. Date: Dec. 23, 2004

(54) Inhibiting Coronaviridae viral replication and treating Coronaviridae viral infection with nucleoside compounds

(76) Inventors: David B. Olsen, Lansdale, PA (US); Joanne E. Tomassini, L. Gwynedd, PA (US); Shi-Shan Mao, North Wales, PA (US); Steven S. Carroll, Yardley, PA (US)

Correspondence Address: MERCK AND CO INC, P O BOX 2000, RAHWAY, NJ, 070650907

(21) Appl. No.: 10/832,945

(22) Filed: Apr. 27, 2004

Related U.S. Application Data
(60) Provisional application No. 60/467,068, filed on May 1, 2003.
(60) Provisional application No. 60/470,658, filed on May 15, 2003.

(51) Int. Cl. A61K 31/381; A61K 31/365

(52) U.S. Cl. 514/447; 514/473

(57) ABSTRACT

Infection by a Coronaviridae virus (e.g., a coronavirus) and/or illness due to a Coronaviridae virus are treated or protected against by administration of a therapeutically or prophylactically effective amount of certain nucleoside compounds and derivatives thereof, either alone or in a composition comprising the nucleoside compound or its derivative and a pharmaceutically acceptable carrier. In addition, replication of a Coronaviridae virus is inhibited by administration of the nucleoside compounds and derivatives thereof, either alone or in pharmaceutical compositions. The nucleosides are particularly suitable for use in treating or prophylaxis of an infection by the SARS virus and/or in treating or prophylaxis of SARS, and for use in inhibiting replication of the SARS virus. The nucleoside compounds and derivatives can optionally be administered in combination with other agents active against the Coronaviridae virus and/or an illness due to the virus. The nucleoside compounds are also for use in the manufacture of medicaments for the inhibition of Coronaviridae virus replication, for the treatment or prophylaxis of Coronaviridae virus infection, and/or for the treatment or prophylaxis of an illness due to a Coronaviridae virus (e.g., the SARS virus). In addition, the compounds are for use as medicaments for the inhibition of Coronaviridae virus replication, for the treatment or prophylaxis of Coronaviridae virus infection, and/or for the treatment or prophylaxis of an illness due to a Coronaviridae virus.

### FIELD OF THE INVENTION

The present invention is directed to methods for inhibiting the replication of Coronaviridae viruses, methods for treating or prophylaxis of infections by Coronaviridae viruses, and methods for treating or prophylaxis of illnesses due to Coronaviridae viruses with certain nucleoside compounds and derivatives thereof. The compounds and derivatives thereof are particularly useful for inhibiting replication of the virus responsible for severe acute respiratory syndrome (SARS), for treatment or prophylaxis of infection due to the SARS virus, and for treatment or prophylaxis of SARS. The present invention is also directed to the use of the nucleoside compounds and derivatives thereof for the manufacture of medicaments for the inhibition of Coronaviridae virus replication and/or for the treatment or prophylaxis of Coronaviridae virus infection or an illness due to Coronaviridae virus infection. The present invention is further directed to use of the nucleoside compounds and derivatives thereof as medicaments for the inhibition of Coronaviridae virus replication and/or for the treatment or prophylaxis of Coronaviridae virus infection or an illness due to Coronaviridae virus infection.

### BACKGROUND OF THE INVENTION

Viruses of the family Coronaviridae are positive strand RNA viruses that typically cause respiratory and enteric diseases in humans and/or domestic animals. The Coronaviridae family includes two genera: coronavirus and torovirus. Coronaviruses and toroviruses have many common features with respect to their genome organization and mode of replication, but differ in virion morphology and genome length. Coronaviruses are typically of more interest because they are more often associated with human infection than are toroviruses. In addition to causing respiratory and enteric disease, coronaviruses have been associated with pneumonia, exacerbation of asthma, neurological symptoms and myocarditis (K. V. Holmes, “Coronaviruses”, in D. Knipe and P. Howley, (ed.), Fields Virology, 4th edition, Lippincott, Williams, and Wilkins. Philadelphia, Pa., 2001, pp. 1187-1203). A novel coronavirus has recently been identified as the etiological agent of severe acute respiratory syndrome (SARS) (Ksiazek et al., The New Eng. J. of Med 2003, 348(20): 1947-1958; Drosten et al, The New Eng. J. of Med. 2003, 348(20): 1959-1968), a life-threatening and highly transmissible disease of such magnitude that it has been declared a quarantinable communicable disease in the United States and by The World Health Organization (WHO) (“Severe Acute Respiratory Syndrome (SARS) and Coronavirus Testing—United States, 2003”, Morbidity and Mortality Weekly Report, Apr. 11, 2003, 52(14): 297-302.). SARS is increasing in epidemic proportions in China, and over 4600 cases were reported worldwide within months of it discovery, with 10% of the cases presenting as severe infections and 5.8% of the cases resulting in death (“Cumulative Number of Reported Probable Cases of Severe Acute Respiratory Syndrome (SARS)”, from November 2002 to 25 Apr. 2003, 17:00 GMT+2, World Health organization, http//www.who.int/en/).

The prototypical coronaviruses which have been characterized include mouse hepatitis virus (MHV), avian infectious bronchitis virus (IBV), porcine transmissible gastroenteritis virus (TGEV), bovine coronavirus (BCV), human coronavirus (HCoV) and feline infectious peritonitis virus (FIPV). The coronaviruses are enveloped viruses, containing single-stranded RNA genomes of positive sense polarity, 27-32 kb in size, which are the largest of known RNA virus genomes (K. V. Holmes, “Coronaviruses”, see full cite above). The RNAs are polyadenylated and 5′ capped, and following entry into the host cell are translated into large polyproteins which are proteolytically cleaved by viral proteinases to yield the viral gene products. The genome encodes the RNA-dependent RNA polymerase (Pol) and four structural proteins common to all coronaviruses, including the spike glycoprotein (S), envelope (E), membrane (M) and nucleocapsid (N) proteins in the order Pol-S-E-M-N. Genomes of MHV also encode a hemagglutinin esterase (HE) glycoprotein located between Pol and S. Additional open reading frames (ORFs) encoding non-structural proteins of unknown and non-conserved function are inserted at various sites in the genome between Pol and S. From within the large 20 kDa Pol gene, two ORFs (orf 1a and 1b) are translated which encode a chymotrypsin-like, cysteine protease and two papain-like proteases responsible for the proteolytic processing of the non-structural proteins. A helicase domain is also found within the Pol gene. Based upon these characteristics, coronavirus replication is predicted to be similar to that of other positive strand RNA viruses.

At present, antiviral drugs for viruses of the Coronaviridae family, especially for coronaviruses, are not available, except for over-the counter-drugs providing symptomatic relief. While coronaviruses generally cause mild to moderate infections of relatively short duration in humans (e.g., “common colds”), the incidence of disease is sufficiently high to be of economic importance, causing losses in school and workplace productivity. More particularly, currently available drugs approved for other viral diseases have so far been found ineffective against the highly transmissible and often fatal SARS. Although veterinary vaccines for the treatment of IBV, TGEV and dog coronavirus (CCoV) are effective in young animals, vaccines to prevent human coronavirus are not available and moreover may not be feasible to develop due to viral heterogeneity and recombination. Given the virulence of coronaviruses in animals (K. V. Holmes, “Coronaviruses”, see full cite above), there is a concern that should SARS proceed unchecked, it could potentially become a far greater global problem than at present. Accordingly, there is an urgent medical need to identify and/or develop antiviral agents effective against the SARS-associated coronavirus. In addition, there is a need to identify and/or develop antiviral agents for Coronaviridae viruses generally for use in the treatment of viral infections, especially coronavirus infections such as the common cold.

The following reference is of interest as background with respect to the present invention:

WO02/057425 discloses nucleoside derivatives useful as inhibitors of hepatitis C virus (HCV) NS5B polymerase, as inhibitors of HCV replication, and/or for the treatment of hepatitis C infection.

### SUMMARY OF THE INVENTION

Certain nucleoside compounds and derivatives thereof have been identified as potent inhibitors of the replication of viruses of the family Coronaviridae and have been determined to be useful in the treatment and prophylaxis of infection by viruses of the Coronaviridae family and in the treatment and prophylaxis of illness due to viruses of the Coronaviridae family. In particular, these nucleoside compounds and their derivatives are active against coronavirus replication, and more particularly against replication of the SARS-associated coronavirus (hereinafter alternatively referred to as the “SARS virus”). Accordingly, the present invention includes a method for inhibiting replication of a Coronaviridae virus, for treating or prophylaxis of an infection by a Coronaviridae virus, or for treating or prophylaxis of an illness due to a Coronaviridae virus in a subject in need thereof, which comprises administering to the subject an inhibition effective amount or a therapeutically or prophylactically effective amount of a compound of Formula I, or a pharmaceutically acceptable salt thereof:

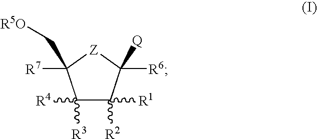

wherein

Z is O or S;

R¹ is H, OH, C₂₋₄ alkenyl, C₂₋₄ alkynyl, C₁₋₄ alkyl-O—, C₁₋₄ alkyl, C₁₋₄ haloalkyl, or C₁₋₄ alkyl substituted with from 1 to 4 substituents each of which is independently OH, amino, C₁₋₄ alkyl-O—, or C₁₋₄ alkyl-S—;

R² is H, OH, amino, halogen, C₁₋₄ alkyl-CH(NH₂)-carbonyloxy, C₁₋₁₆ alkylcarbonyloxy, mercapto, C₁₋₄ alkyl-O—, C₁₋₄ alkyl, C₁₋₄ haloalkyl, or C₁₋₄ alkyl substituted with from 1 to 4 substituents each of which is independently OH, amino, C₁₋₄ alkyl-O—, or C₁₋₄ alkyl-S—;

R³ is H, OH, cyano, azido, halogen, C₁₋₄ alkyl-CH(NH₂)-carbonyloxy, C₁₋₁₆ alkylcarbonyloxy, C₂₋₁₈ alkenylcarbonyloxy, C₄₋₁₈ polyalkenylcarbonyloxy, C₁₋₁₀ alkyloxycarbonyloxy, C₃₋₆ cycloalkylcarbonyloxy, C₃₋₆ cycloalkyloxycarbonyloxy, mercapto, amino, C₁₋₄ alkyl-O—, C₂₋₄ alkenyl, C₂₋₄ alkynyl, C₁₋₄ alkyl, C₁₋₄ haloalkyl, or C₁₋₄ alkyl substituted with from 1 to 4 substituents each of which is independently OH, amino, C₁₋₄ alkyl-O—, or C₁₋₄ alkyl-S—;

R⁴ is H, OH, cyano, azido, halogen, C₁₋₁₆ alkylcarbonyloxy, C₂₋₁₈ alkenylcarbonyloxy, C₄₋₁₈ polyalkenylcarbonyloxy, C₁₋₁₀ alkyloxycarbonyloxy, C₃₋₆ cycloalkylcarbonyloxy, C₃₋₆ cycloalkyloxycarbonyloxy, mercapto, amino, C₁₋₄ alkyl-O—, C₂₋₄ alkenyl, C₂₋₄ alkynyl, C₁₋₄ alkyl, C₁₋₄ haloalkyl, or C₁₋₄ alkyl substituted with from 1 to 4 substituents each of which is independently OH, amino, C₁₋₄ alkyl-O—, or C₁₋₄ alkyl-S—;

R⁵ is H, C₁₋₄ alkyl-CH(NH₂)-carbonyloxy, C₁₋₁₆ alkylcarbonyl, C₂₋₁₈ alkenylcarbonyl, C₄₋₁₈ polyalkenylcarbonyloxy, C₁₋₁₀ alkyloxycarbonyl, C₃₋₆ cycloalkylcarbonyl, C₃₋₆ cycloalkyloxycarbonyl, P₃O₉H₄, P₂O₆H₃, or P(O)R^{u}R^{v};

R⁶ is H, methyl, hydroxymethyl, or fluoromethyl;

R⁷ is H, methyl, hydroxymethyl, fluoromethyl, aminomethyl, azido, or cyano;

Q is:

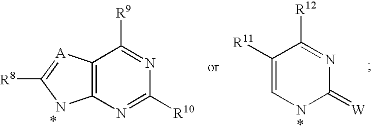

wherein

* denotes the point of attachment of Q to the C-1 carbon of the furanose ring;

A is N or C—R^{w};

W is O or S;

R⁸ is H, C₁₋₄ alkyl, C₂₋₄ alkynyl, halogen, cyano, carboxy, C₁₋₄ alkyloxycarbonyl, azido, amino, C₁₋₄ alkylamino, di(C₁₋₄ alkyl)amino, OH, C₁₋₆ alkyl-O—, C₁₋₆ alkyl-S—, C₁₋₆ alkyl-SO₂—, aminomethyl, or (C₁₋₄ alkyl)₁₋₂aminomethyl;

R⁹ and R¹² are each independently H, OH, mercapto, halogen, C₁₋₄ alkyl-O—, C₁₋₄ alkyl-S—, C₁₋₈ alkylcarbonyloxy, C₃₋₆ cycloalkylcarbonyloxy, C₁₋₈ alkyloxycarbonyloxy, C₃₋₆ cycloalkyloxycarbonyloxy, —OCH₂CH₂SC(═O)C₁₋₄ alkyl, —OCH₂OC(═O)C₁₋₄ alkyl, —OCH(C₁₋₄ alkyl)OC(═O)C₁₋₄ alkyl, amino, C₁₋₄ alkylamino, di(C₁₋₄ alkyl)amino, C₃₋₆ cycloalkylamino, di(C₃₋₆ cycloalkyl)amino, or an amino acyl residue of formula:

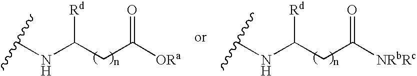

wherein n is an integer equal to zero, 1, 2, 3 or 4;

R¹⁰ is H, OH, mercapto, halogen, C₁₋₄ alkyl-O—, C₁₋₄ alkyl-S—, amino, C₁₋₄ alkylamino, di(C₁₋₄ alkyl)amino, C₃₋₆ cycloalkylamino, di(C₃₋₆ cycloalkyl)amino, phenyl-C₁₋₂ alkylamino, C₁₋₄ alkyl-C(═O)NH—, C₁₋₈ alkylcarbonyloxy, or —OCH(C₁₋₄ alkyl)OC(═O)C₁₋₄ alkyl;

R¹¹ is H, C₁₋₆ alkyl, C₂₋₆ alkenyl, C₂₋₆ alkynyl, C₁₋₄ alkylamino, CF₃, or halogen;

R^{a}, R^{b}, and R^{c} are each independently H or C₁₋₆ alkyl;

R^{d} is H, C₁₋₄ alkyl, phenyl-C₁₋₂ alkyl, or phenyl;

R^{u} and R^{v} are each independently OH, —OCH₂CH₂SC(═O)C₁₋₄ alkyl, —OCH₂OC(═O)OC₁₋₄ alkyl, —NHCHMeCO₂Me, —OCH(C₁₋₄ alkyl)OC(═O)C₁₋₄ alkyl,

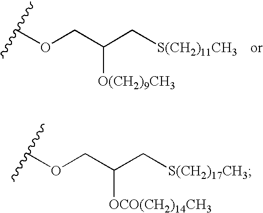

R^{w} is H, cyano, nitro, NHC(═O)NH₂, C(═O)NR^{x}R^{x}, CSNR^{x}R^{x}, C(═O)OR^{x}, C(═NH)NH₂, OH, C₁₋₃ alkoxy, amino, C₁₋₄ alkylamino, di(C₁₋₄ alkyl)amino, halogen, C₁₋₃ alkyl, or C₁₋₃ alkyl substituted with from one to three groups independently selected from halogen, amino, OH, carboxy, and C₁₋₃ alkyl-O—; and

each R^{x} is independently H or C₁₋₆ alkyl.

An embodiment of the present invention is the method as just described, wherein the Coronaviridae virus is a coronavirus. Another embodiment of the present invention is the method as originally described, wherein the Coronaviridae virus is the SARS virus. In an aspect of each of these embodiments, the subject is a human.

The compounds of Formula I are believed to be inhibitors of RNA-dependent RNA viral polymerase which is conserved among Coronaviridae viruses, and thus are believed to be inhibitors of RNA-dependent RNA viral replication.

Other embodiments, aspects and features of the present invention are either further described in or will be apparent from the ensuing description, examples and appended claims.

### DETAILED DESCRIPTION OF THE INVENTION

The present invention includes a method for inhibiting replication of a Coronaviridae virus, for treating or prophylaxis of an infection by a Coronaviridae virus, or for treating or prophylaxis of an illness due to a Coronaviridae virus in a subject in need thereof, which comprises administering to the subject an inhibition effective amount or a therapeutically or prophylactically effective amount of a compound of Formula I, or a pharmaceutically acceptable salt thereof, as set forth above in the Summary of the Invention.

A first embodiment of the present invention is the method as originally defined above, except that in the compound of Formula I (hereinafter alternatively referred to as “Compound I”):

Z is 0;

R¹ is H, OH, C₁₋₃ alkyl-O—, C₁₋₃ alkyl, C₁₋₃ haloalkyl, or C₁₋₃ alkyl mono-substituted with OH, amino, C₁₋₄ alkyl-O— or C₁₋₄ alkyl-S—;

R² is H, OH, amino, fluoro, C₁₋₄ alkyl-CH(NH₂)-carbonyloxy, C₁₋₁₆ alkylcarbonyloxy, mercapto, C₁₋₃ alkyl-O—, C₁₋₃ alkyl, or C₁₋₃ haloalkyl;

R³ is H, OH, halogen, C₁₋₄ alkyl-CH(NH₂)-carbonyloxy, C₁₋₁₆ alkylcarbonyloxy, amino, C₁₋₃ alkyl-O—, C₁₋₃ alkyl, or C₁₋₃ haloalkyl;

R⁴ is H, OH, halogen, C₁₋₁₆ alkylcarbonyloxy, amino, C₁₋₃ alkyl-O—, C₁₋₃ alkyl, or C₁₋₃ haloalkyl;

R⁵ is H, C₁₋₄ alkyl-CH(NH₂)-carbonyl, C₁₋₆ alkylcarbonyl, P₃O₉H₄, P₂O₆H₃, or PO₃H₂;

R⁶ and R⁷ are both H;

and all other variables are as originally defined.

A second embodiment of the present invention is the method as originally defined above, except that in Compound I:

Z is O;

R¹ is H, OH, methyl, methoxy, fluoromethyl, hydroxymethyl, difluoromethyl, trifluoromethyl, or aminomethyl;

R² is H, OH, fluoro, (CH₃)₂CHCH(NH₂)-carbonyloxy, C₁₋₁₆ alkylcarbonyloxy, or methoxy;

R³ is H, OH, fluoro, (CH₃)₂CHCH(NH₂)-carbonyloxy, C₁₋₁₆ alkylcarbonyloxy, amino, or methoxy;

R⁴ is H;

R⁵ is H, (CH₃)₂CHCH(NH₂)-carbonyl, C₁₋₁₆ alkylcarbonyl, or P₃O₉H₄;

R⁶ and R⁷ are both H;

and all other variables are as originally defined.

In an aspect of the second embodiment, R¹ is methyl; and R² and R³ are both OH. In another aspect of the second embodiment, R² and R³ are each independently OH or (CH₃)₂CHCH(NH₂)-carbonyloxy; and R⁵ is H or (CH₃)₂CHCH(NH₂)-carbonyl. In a feature of this aspect, R¹ is methyl. In another feature of this aspect, Compound I is selected from the group consisting of:

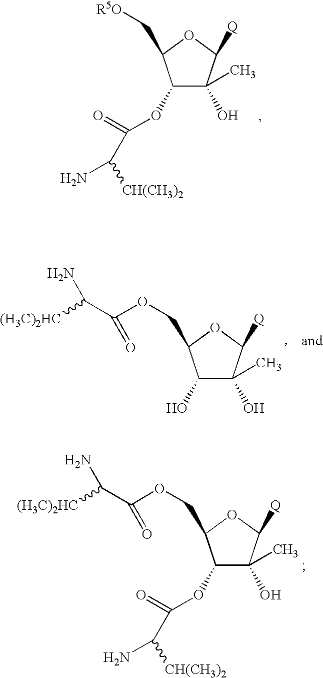

wherein Q is as originally defined.

A third embodiment of the present invention is the method as originally defined above, except that in Compound I:

Q is:

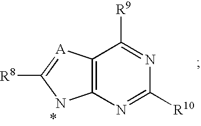

A is N or C—R^{w};

R⁸ is H, C₁₋₃ alkyl, halogen, azido, amino, C₁₋₄ alkylamino, or C₁₋₃ alkyl-O—;

R⁹ and R¹⁰ are each independently H, OH, halogen, amino, C₁₋₄ alkylamino, di(C₁₋₄ alkyl)amino, or C₃₋₆ cycloalkylamino;

R^{w} is hydrogen, cyano, methyl, halogen, or C(═O)NH₂;

and all other variables are as originally defined.

A fourth embodiment of the present invention is the method as originally defined, except that the compound of Formula I is a compound of Formula H (or “Compound II”), or a pharmaceutically acceptable salt thereof:

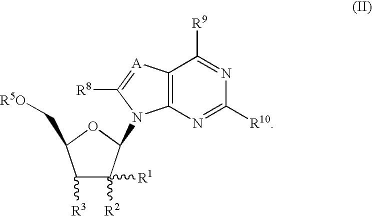

wherein A, R¹, R², R³, R⁵, R⁸, R⁹, and R¹⁰ are as originally defined.

A fifth embodiment of the present invention is the method as originally defined, except that the compound of Formula I is Compound II, wherein

A is N or C—R^{w};

R⁸ is H, C₁₋₃ alkyl, halogen, azido, amino, C₁₋₄ alkylamino, or C₁₋₃ alkyl-O—;

R⁹ and R¹⁰ are each independently H, OH, halogen, amino, C₁₋₄ alkylamino, di(C₁₋₄ alkyl)amino, or C₃₋₆ cycloalkylamino;

R^{w} is hydrogen, cyano, methyl, halogen, or C(═O)NH₂;

and all other variables are as originally defined.

A sixth embodiment of the present invention is the method as originally defined above, except that the compound of Formula I is Compound II, wherein

R¹ is H, OH, methyl, methoxy, fluoromethyl, hydroxymethyl, difluoromethyl, trifluoromethyl, or aminomethyl;

R² is H, OH, fluoro, C₁₋₁₆ alkylcarbonyloxy, or methoxy;

R³ is H, OH, fluoro, (CH₃)₂CHCH(NH₂)-carbonyloxy, C₁₋₁₆ alkylcarbonyloxy, amino, or methoxy;

R⁵ is H, (CH₃)₂CHCH(NH₂)-carbonyl, C₁₋₁₆ alkylcarbonyl, or P₃O₉H₄;

A is Nor C—R^{w};

R⁸ is H, C₁₋₃ alkyl, halogen, azido, amino, C₁₋₄ alkylamino, or C₁₋₃ alkyl-O—;

R⁹ and R¹⁰ are each independently H, OH, halogen, amino, C₁₋₄ alkylamino, di(C₁₋₄ alkyl)amino, or C₃₋₆ cycloalkylamino; and

R^{w} is hydrogen, cyano, methyl, halogen, or C(═O)NH₂.

In an aspect of the sixth embodiment, R¹ is methyl; R² and R³ are both OH; and R⁵ is H, (CH₃)₂CHCH(NH₂)-carbonyl, C₁₋₁₆ alkylcarbonyl, or P₃O₉H₄.

A seventh embodiment of the present invention is the method as originally defined above, wherein Compound I is a compound selected from the group consisting of:

4-amino-7-(2-C-methyl-β-D-ribofuranosyl)-7H-pyrrolo[2,3-d]pyrimidine;

2′-C-methyladenosine;

corresponding 5′-triphosphates thereof;

and pharmaceutically acceptable salts thereof.

An eighth embodiment of the present invention is the method as originally defined above, wherein Compound I is 4-amino-7-(2-C-methyl-β-D-ribofuranosyl)-7H-pyrrolo[2,3-d]pyrimidine, or a pharmaceutically acceptable salt thereof.

A ninth embodiment of the present invention is the method as originally defined above, except that in Compound I:

Q is

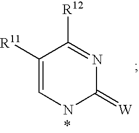

W is O;

R¹¹ is H, C₁₋₃ alkyl, C₁₋₃ alkylamino, or halogen;

R¹² is H, OH, halogen, amino, C₁₋₄ alkylamino, di(C₁₋₄ alkyl)amino, or C₃₋₆ cycloalkylamino;

and all other variables are as originally defined.

A tenth embodiment of the present invention is the method as originally defined above, except that in Compound I:

Z is O;

R¹ is H, OH, methyl, methoxy, fluoromethyl, hydroxymethyl, difluoromethyl, trifluoromethyl, or aminomethyl;

R² is H, OH, fluoro, C₁₋₁₆ alkylcarbonyloxy, or methoxy;

R³ is H, OH, fluoro, (CH₃)₂CHCH(NH₂)-carbonyloxy, C₁₋₁₆ alkylcarbonyloxy, amino, or methoxy;

R⁴ is H;

R⁵ is H, (CH₃)₂CHCH(NH₂)-carbonyl, C₁₋₆ alkylcarbonyl, or P₃O₉H₄; and

R⁶ and R⁷ are both H;

Q is

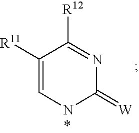

W is O;

R¹¹ is H, C₁₋₃ alkyl, C₁₋₃ alkylamino, or halogen; and

R¹² is H, OH, halogen, amino, C₁₋₄ alkylamino, di(C₁₋₄ alkyl)amino, or C₃₋₆ cycloalkylamino.

In an aspect of the tenth embodiment, R₁ is methyl; and R² and R³ are both OH. In another aspect of the tenth embodiment, R³ is OH or (CH₃)₂CHCH(NH₂)-carbonyloxy; and R⁵ is H or (CH₃)₂CHCH(NH₂)-carbonyl. In a feature of this aspect, R₁ is methyl.

An eleventh embodiment of the present invention is the method as originally defined above, wherein Compound I is 2′-C-methylcytidine, or a pharmaeutically acceptable salt thereof.

Other embodiments of the present invention include the method as originally defined above or as defined in any of the foregoing embodiments, or in an aspect or feature thereof, incorporating one or more of the following additional features (a), (b) and (c):

(a1) the method is a method for inhibiting replication of a Coronaviridae virus;

(a2) the method is a method for treating or prophylaxis of infection by a Coronaviridae virus;

(a3) the method is a method for treating or prophylaxis of an illness due to a Coronaviridae virus;

(b1) the Coronaviridae virus is a coronavirus;

(b2) the Coronaviridae virus is the SARS virus;

(c1) the compound of Formula I is administered in combination with a therapeutically effective amount of another agent active against the Coronaviridae virus;

(c2) the compound of Formula I is administered in combination with a therapeutically effective amount of another agent active against the Coronaviridae virus, wherein the other agent is interferon, ribavirin, levovirin, or viramidine; and

(c3) the compound of Formula I is administered in combination with a therapeutically effective amount of another agent active against the Coronaviridae virus, wherein the other agent is an angiotensin II receptor blocker (e.g., losartan).

The present invention also includes the method as originally set forth above and as defined in the embodiments, aspects and features thereof, wherein one compound encompassed by Formula I is administered in combination with at least one other compound encompassed by Formula I. The co-administration of two or more compounds of Formula I can result in a synergistic effect with respect to inhibition of replication of a Coronaviridae virus, treatment or prophylaxis of infection by a Coronaviridae virus, and/or treatment or prophylaxis of an illness due to a Coronaviridae virus. More particularly, co-administration of two or more nucleoside compounds of Formula I can result in a synergistic effect with respect to inhibition of replication of the SARS virus, treatment or prophylaxis of infection by the SARS virus, and/or treatment or prophylaxis of SARS.

The present invention also includes a compound of Formula I for: (i) use in, (ii) use as a medicament for, or (iii) use in the preparation of a medicament for: (a) inhibiting replication of a Coronaviridae virus, (b) for treating or prophylaxis of an infection by a Coronaviridae virus, or (c) for treating or prophylaxis of an illness due to a Coronaviridae virus. In these uses, the compounds of Formula I can optionally be employed in combination with one or more agents active against a Coronaviridae virus or against an illness due to a Coronaviridae virus. Additional embodiments of the invention include any of the uses set forth in the first sentence of this paragraph, wherein the nucleoside compound of Formula I employed therein is a compound as defined in one of the embodiments, aspects, or features of the method of the present invention set forth above. In all of these embodiments, the compound may optionally be used in the form of a pharmaceutically acceptable salt.

The present invention also includes a method of inhibiting SARS virus replication in a human in need of such inhibition, which comprises administering to the human an inhibition effective amount of Compound A, or a pharmaceutically acceptable salt thereof; wherein Compound A is 4-amino-7-(2-C-methyl-β-D-ribofuranosyl)-7H-pyrrolo[2,3-d]pyrimidine. In an embodiment of this method, Compound A is administered in combination with an inhibition effective amount of another agent active against the SARS virus. In an aspect of this embodiment, the other agent active against the SARS virus is interferon, ribavirin, levovirin, or viramidine. In another aspect of this embodiment, the other agent active against the SARS virus is an angiotensin II receptor blocker (e.g., losartan). In still another aspect of this embodiment, the other agent active against the SARS virus is 2′-C-methylcytidine, or a pharmaceutically acceptable salt thereof.

The present invention also includes a method for treating or prophylaxis of a SARS virus infection in a human in need of such treatment or prophylaxis, which comprises administering to the human a therapeutically or prophylactically effective amount of Compound A, or a pharmaceutically acceptable salt thereof. In an embodiment of this method, Compound A is administered in combination with a therapeutically or prophylactically effective amount of another agent active against SARS virus infection. In an aspect of this embodiment, the other agent active against SARS virus infection is interferon, ribavirin, levovirin, or viramidine. In another aspect of this embodiment, the other agent active against the SARS virus is an angiotensin II receptor blocker (e.g., losartan). In still another aspect of this embodiment, the other agent active against the SARS virus is 2′-C-methylcytidine, or a pharmaceutically acceptable salt thereof.

The present invention also includes a method for treating or prophylaxis of SARS in a human in need of such treatment, which comprises administering to the human a therapeutically or prophylactically effective amount of Compound A, or a pharmaceutically acceptable salt thereof. In an embodiment of this method, Compound A is administered in combination with a therapeutically or prophylactically effective amount of another agent active against SARS. In an aspect of this embodiment, the other agent active against SARS is interferon, ribavirin, levovirin, or viramidine. In another aspect of this embodiment, the other agent active against the SARS virus is an angiotensin II receptor blocker (e.g., losartan). In still another aspect of this embodiment, the other agent active against the SARS virus is 2′-C-methylcytidine, or a pharmaceutically acceptable salt thereof.

The term “subject” as used herein refers to any vertebrate species which is the object of treatment, observation or experiment with respect to the present invention. In one embodiment, the subject is a warm-blooded vertebrate, particularly a mammal, preferably a primate, and more preferably a human.

The term “treating”, or a variant thereof (e.g., “treatment”), refers to reducing or ameliorating an existing undesirable or adverse condition, symptom or disease (e.g., SARS) or delaying its onset in a subject in need of such reduction, amelioration or delay.

The term “prophylaxis” refers to protecting a subject from an undesirable or adverse condition, symptom or disease, wherein the subject typically has or will have an increased risk of acquiring such a condition, symptom, or disease as a result of actual, suspected or anticipated exposure to the causative agent thereof (e.g., a coronavirus). “Increased risk” means a statistically higher frequency of occurrence of the condition, symptom, or disease in the subject as a result of exposure to the causative agent in comparison to the frequency of its occurrence in the general population. For example, healthcare workers serving SARS patients would be at a substantially increased risk for acquiring SARS relative to the general population. Prophylaxis includes administration of an effective amount of the nucleoside compound or its derivative to a subject (e.g., a healthcare worker) who will have potential exposure to the causative agent (e.g., a Coronaviridae virus), for a period of time before, during, and after the potential exposure sufficient to prevent development of an undesirable or adverse condition, symptom, or disease that can result from actual exposure. Prophylaxis also includes administration of an effective amount of the nucleoside compound or its derivative to a subject who has already been exposed to the causative agent but has not yet exhibited an undesirable condition, symptom or disease, for a time sufficient to prevent development of the undesirable condition, symptom or disease.

The term “therapeutically effective amount” means that amount of active agent or active ingredient that elicits the biological or medicinal response in a tissue, system, animal or human that is being sought by a researcher, veterinarian, physician or other clinician, which includes alleviation of the symptoms of the disease or condition being treated.

The term “prophylactically effective amount” means that amount of active agent or active ingredient that elicits the biological or medicinal response in a tissue, system, animal or human that is being sought by a researcher, veterinarian, physician or other clinician, which includes prevention of the symptoms of the disease or condition being treated.

As used herein, the term “alkyl” refers to any linear or branched chain alkyl group having a number of carbon atoms in the specified range. Thus, for example, “C₁₋₆ alkyl” (or “C₁-C₆ alkyl”) refers to all of the hexyl alkyl and pentyl alkyl isomers as well as n-, iso-, sec- and t-butyl, n- and isopropyl, ethyl and methyl. As another example, “C₁₋₄ alkyl” refers to n-, iso-, sec- and t-butyl, n- and isopropyl, ethyl and methyl.

The term “alkenyl” means any linear or branched chain alkenyl group having a number of carbon atoms in the specified range. Thus, for example, “C₂₋₆ alkenyl” (or “C₂-C₆ alkenyl”) refers to all of the hexenyl and pentenyl isomers as well as 1-butenyl, 2-butenyl, 3-butenyl, isobutenyl, 1-propenyl, 2-propenyl, and ethenyl (or vinyl).

The term “alkynyl” means any linear or branched chain alkynyl group having a number of carbon atoms in the specified range. Thus, for example, “C₂₋₆ alkynyl” (or “C₂-C₆ alkynyl”) refers to all of the hexynyl and pentynyl isomers as well as 1-butynyl, 2-butynyl, 3-butynyl, 1-propynyl, 2-propynyl, and ethynyl (or acetylenyl).

The term “cycloalkyl” means any cyclic ring of an alkane having a number of carbon atoms in the specified range. Thus, for example, “C₃₋₆ cycloalkyl” (or “C₃-C₆ cycloalkyl”) refers to cyclopropyl, cyclobutyl, cyclopentyl, and cyclohexyl.

The term “halogen” (or “halo”) refers to fluorine, chlorine, bromine and iodine (alternatively referred to as fluoro, chloro, bromo, and iodo).

The term “haloalkyl” refers to an alkyl group as defined above in which one or more of the hydrogen atoms has been replaced with a halogen (i.e., F, Cl, Br and/or I). Thus, for example, “C₁₋₆ haloalkyl” (or “C₁-C₆ haloalkyl”) refers to a C₁ to C₆ linear or branched alkyl group as defined above with one or more halogen substituents. The term “C₁₋₆ fluoroalkyl” has an analogous meaning except that the halogen substituents are restricted to fluoro. Suitable fluoroalkyls include the series (CH₂)₀₋₄CF₃ (i.e., trifluoromethyl, 2,2,2-trifluoroethyl, 3,3,3-trifluoro-n-propyl, etc.).

The term “alkylamino” refers to any straight or branched alkylamine having the specified number of carbon atoms. Thus, for example, “C₁₋₄ alkylamino” refers to methylamino (CH₃NH—), ethylamino (CH₃CH₂NH—), n-propylamino (CH₃CH₂CH₂NH—), isopropylamino (CH₃)₂CHNH—), and n-, iso-, sec- and t-butylamino.

The term “alkyloxycarbonyl” refers to straight or branched chain esters of a carboxylic acid derivative having the specified number of carbon atoms. Thus, for example, “C₁₋₄ alkyloxycarbonyl” refers to methyloxycarbonyl (MeOC(═O)—), ethyloxycarbonyl (EtOC(═O)—), n-propyloxycarbonyl (CH₃CH₂CH₂OC(═O)—), isopropyloxycarbonyl (CH₃)₂CHOC(═O)—), and n-, iso-, sec- and t-butyloxycarbonyl.

The term “alkyloxycarbonyloxy” refers to straight or branched chain alkyl carbonates having the specified number of carbon atoms. Thus, for example, “C₁₋₁₀ alkyloxycarbonyloxy)” refers to ROC(═O)O— in which R is a C₁₋₁₀ alkyl group, such as methyloxycarbonyloxy (MeOC(═O)O—), ethyloxycarbonyloxy (EtOC(═O)O—), or t-butyloxycarbonyloxy ((CH₃)₃COC(═O)O—).

The term “alkylcarbonyloxy” refers to straight or branched chain alkylcarboxylic acid groups having the specified number of carbon atoms. Thus, for example, “C₁₋₁₆ alkylcarbonyloxy” refers to RC(═O)O— in which R is a C₁₋₁₆ alkyl group, such as methylcarbonyloxy (MeC(═O)O—), ethylcarbonyloxy (EtC(═O)O—), or n-octylcarbonyloxy (CH₃(CH₂)₆CH₂C(═O)O—).

The term “cycloalkylcarbonyloxy” refers to cyclic alkanoic acid groups having the specified number of carbon atoms. Thus, for example, “C₃₋₆ cycloalkylcarbonyloxy” refers to cyclopropylcarbonyloxy, cyclopentylcarbonyloxy, or cyclohexylcarbonyloxy.

The term “alkenylcarbonyloxy” refers to a straight or branched chain alkenoic acid group having the specified number of carbon atoms. Thus, for example, “C₂₋₁₈ alkenylcarbonyloxy” refers to RC(═O)O— in which R is a C₂₋₁₈ alkenyl group, such as ethenylcarbonyloxy (CH₂=CH₂C(═O)O—) or 1-propen-3-yl-carbonyloxy ((CH₂═CH₂CH₂C(═O)O—).

The term “polyalkenylcarbonyloxy” refers to a straight or branched chain alkadienoic or alkatrienoic acid group having the specified number of carbon atoms. Thus, for example, “C₄₋₁₈ polyalkenylcarbonyloxy” refers to RC(═O)O— in which R is a C₄₋₁₈ alkadienyl or alkantrienyl group, such as 1,3-butadien-4-ylcarbonyloxy (CH₂=CH—CH═CH—C(═O)O—).

The term “substituted” (e.g., as in “C₁₋₄ alkyl substituted with from 1 to 4 substituents . . . ”) includes mono- and poly-substitution by a named substituent to the extent such single and multiple substitution (including multiple substitution at the same site) is within the range specified and is chemically allowed.

The term “P₃O₉H₄” (also referred to as the “5′-triphosphate”) appearing in the definition of R⁵ refers to a nucleoside compound of Formula I which is a triphosphoric acid ester derivative of the 5′-hydroxyl group thereof, and more particularly refers to a compound of Formula III:

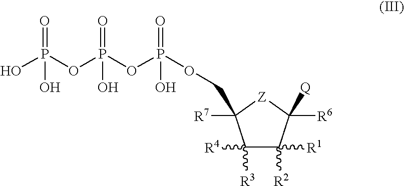

wherein Q, Z, R¹—R⁴, R⁶ and R⁷ are as defined above.

The term “P₂O₆H₃” (also referred to as the “5“-diphosphate”) appearing in the definition of R⁵ refers to a nucleoside compound of Formula I which is a diphosphoric acid ester derivative of the 5′-hydroxyl group thereof, and more particularly refers to a compound Formula IV:

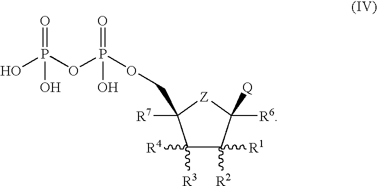

wherein Q, Z, R¹—R⁴, R⁶ and R⁷ are as defined above.

The term “P(O)R^{u}R^{v}” appearing in the definition of R⁵ refers to “PO₃H₂” (or “5′-monophosphate”) when R^{u}═R^{v}═OH, which is a monophosphoric acid ester derivative of the 5′-hydroxyl group in the nucleoside compound of Formula I, and more particularly refers to a compound Formula V:

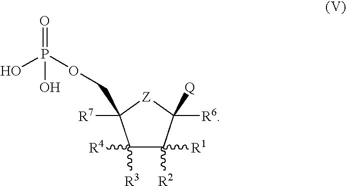

wherein Q, Z, R¹—R⁴, R⁶ and R⁷ are as defined above.

The present invention can employ the 5′-triphosphate, 5′-diphosphate, and the 5′-monophosphate as set forth in Formulas III, IV and V respectively as well as pharmaceutically acceptable salts thereof.

The compounds of Formula I employed in the present invention contain one or more asymmetric centers and can thus occur as racemates and racemic mixtures, single enantiomers, diastereomeric mixtures and individual diastereomers. The present invention encompasses administration of compounds of Formula I as racemic mixtures, single enantiomers, diastereomeric mixtures and individual diastereomers. The present invention includes administration of nucleoside compounds having the β-D stereochemical configuration for the five-membered furanose ring as depicted in the structural formula below; that is, nucleoside compounds in which the substituents at C-1 and C-4 of the five-membered furanose ring have the β-stereochemical configuration (“up” orientation as denoted by a bold line).

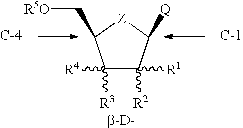

The stereochemistry of the substituents at the C-2 and C-3 positions of the furanose ring of the compounds encompassed by Formula I is denoted by squiggly lines (i.e., “ [symbol] ”) which signifies that substituents R¹, R², R³ and R⁴ can have either the I (substituent “down”) or θ (substituent “up”) configuration independently of one another. Notation of stereochemistry by a bold line as at C-1 and C-4 of the furanose ring signifies that the substituent has the β-configuration (substituent “up”).

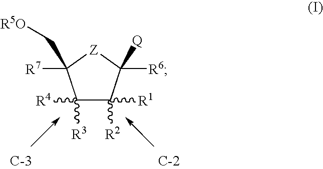

Compounds of Formula I may be separated into their individual diastereomers by, for example, fractional crystallization from a suitable solvent, for example methanol or ethyl acetate or a mixture thereof, or via chiral chromatography using an optically active stationary phase.

Alternatively, any stereoisomer of a compound of the Formula I may be obtained by stereospecific synthesis using optically pure starting materials or reagents of known configuration.

Some of the compounds described herein contain olefinic double bonds, and unless specified otherwise, the present invention includes administration of the E and Z geometric isomers, separately or in mixtures.

Some of the compounds encompassed by Formula I can exist as tautomers such as keto-enol tautomers. The present invention includes administration of individual tautomers as well as tautomeric mixtures. As an example, keto-enol tautomers which are within the scope of compounds of Formula I administered in the methods of the present invention include the following:

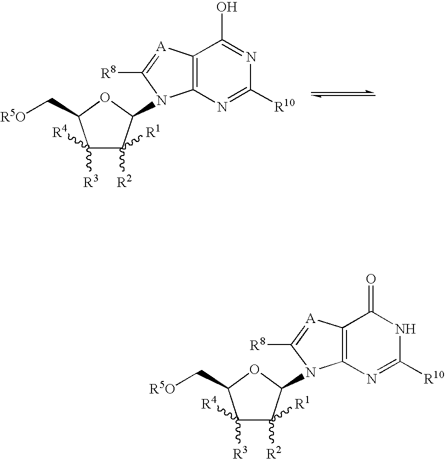

The compounds of Formula I employed in the present invention can be administered in the form of pharmaceutically acceptable salts. The term “pharmaceutically acceptable salt” refers to a salt which possesses the effectiveness of the parent compound and which is not biologically or otherwise undesirable (e.g., is neither toxic nor otherwise deleterious to the recipient thereof). Suitable salts include acid addition salts which may, for example, be formed by mixing a solution of the a compound of Formula I with a solution of a pharmaceutically acceptable organic or inorganic acid such as hydrochloric acid, sulfuric acid, acetic acid, trifluoroacetic acid, or benzoic acid. When the compound of Formula I carries an acidic moiety, suitable pharmaceutically acceptable salts thereof can include those derived from organic or inorganic bases such as alkali metal salts (e.g., sodium or potassium salts), alkaline earth metal salts (e.g., calcium or magnesium salts), and salts formed with suitable organic ligands such as quaternary ammonium salts. Also, in the case of an acid (—COOH) or alcohol group being present, pharmaceutically acceptable esters can be employed to modify the solubility or hydrolysis characteristics of the compound, such as for use in a sustained-release or prodrug formulation.

The present invention includes administration of a compound of Formula I,

or a pharmaceutically acceptable salt thereof, in combination with one or more other agents useful for inhibiting replication of a Coronaviridae virus, for treating or prophylaxis of infection by a Coronaviridae virus, or for treating or prophylaxis of an illness due to a Coronaviridae virus. Agents active against Coronaviridae viruses include, but are not limited to interferon, ribavirin, levovirin, and viramidine. Interferons (IFNs) are a well known family of cytokines secreted by a large variety of eukaryotic cells upon exposure to various stimuli. The interferons have been classified by their chemical and biological characteristics into four groups: IFN-α (leukocytes), IFN-β (fibroblasts), IFN-γ (lymphocytes), and IFN-ω (leukocytes). Interferons (e.g., IFN-α) are known to be useful in antiviral therapy, in particular for HCV, a positive strand RNA virus. Ribavirin is a nucleoside analog (chemical name-1-β-D-ribofuranosyl-1H-1,2,4-triazole-3-carboxamide) that is available in encapsulated form under the tradename REBETOL® (Schering-Plough). Levovirin is the L-enantiomer of ribavirin which has shown immunomodulatory activity similar to ribavirin. Viramidine represents an analog of ribavirin disclosed in WO 01/60379 (assigned to ICN Pharmaceuticals). In the present invention, the compound of Formula I, or a pharmaceutically acceptable salt thereof, and the one or more other active agents of the combination can be administered separately at different times via the same or different routes of administration, during the course of therapy or concurrently in divided or single combination forms. Accordingly, the present invention is understood as embracing any and all regimes of simultaneous or alternating administration of the active components of the combination.

Ribavirin, levovirin, and viramidine can exert their anti-Coronaviridae virus effects by modulating intracellular pools of guanine nucleotides via inhibition of the intracellular enzyme inosine monophosphate dehydrogenase (IMPDH). IMPDH is the rate-limiting enzyme on the biosynthetic route in de novo guanine nucleotide biosynthesis. Ribavirin is readily phosphorylated intracellularly and the monophosphate derivative is an inhibitor of IMPDH. Thus, inhibition of IMPDH represents another useful target for the discovery of inhibitors of Coronaviridae virus replication. Therefore, the compounds of Formula I can also be administered in combination with an inhibitor of IMPDH, such as VX-497, which is disclosed in WO 97/41211 and WO 01/00622 (assigned to Vertex); another IMPDH inhibitor, such as that disclosed in WO 00/25780 (assigned to Bristol-Myers Squibb); or mycophenolate mofetil [see A. C. Allison and E. M. Eugui, Agents Action 1993, 44 (Suppl.): 165].

Other agents which are suitable for use in combination with the nucleoside compounds of Formula I in the method of the present invention include angiotensin II antagonists (also referred to as angiotensin II receptor blockers or ARBs). Suitable ARBs include candesartan cilexetil, eprosartan, irbesartan, losartan, olmesartan, tasosartan, telmisartan, and valsartan. In one embodiment, the ARB administered in combination with Compound I is losartan. Losartan, which is disclosed in U.S. Pat. No. 5,138,069 (herein incorporated by reference), is available in the form of a potassium salt under the tradename COZAAR®. In another embodiment, the ARB administered in combination with Compound I is a composition containing losartan potassium and hydrochlorothiazide, which is disclosed in U.S. Pat. No. 5,153,197 (herein incorporated by reference) and is available under the tradename HYZAAR®. Reports (“GenoMed Reaffirms Potential Utility of Sartans for SARS”, Apr. 28, 2003, http://www.prnewswire.com; “GenoMed Announces Potential Therapy for SARS”, Apr. 25, 2003, http://www.prnewswire.com) have indicated that the ARBs can act to down modulate the host's immune response to SARS and thereby decrease mortality due to SARS.

The scope of the other agents employed in combination with compounds of Formula I in the methods embraced by the present invention includes in principle any other agent useful for inhibiting replication of a Coronaviridae virus, for treating or prophylaxis of infection by a Coronaviridae virus, or for treating or prophylaxis of an illness due to a Coronaviridae virus. When a compound of Formula I, or a pharmaceutically acceptable salt thereof, is used in combination with a second therapeutic agent active against a Coronaviridae virus, the dose of each active ingredient can be the same as or different from the dose when that active ingredient is used alone.

The present invention also includes the methods and uses as heretofore described in which the compound of Formula I is administered as a pharmaceutical composition comprising the compound and a pharmaceutically acceptable carrier. More particularly, the present invention also includes a method for inhibiting replication of a Coronaviridae virus, for treating or prophylaxis of an infection by a Coronaviridae virus, or for treating or prophylaxis of an illness due to a Coronaviridae virus in a subject in need thereof, which comprises administering to the subject a pharmaceutical composition comprising an inhibition effective amount or a therapeutically or prophylactically effective amount of a compound of Formula I, or a pharmaceutically acceptable salt thereof, and a pharmaceutically acceptable carrier. In an embodiment of this method the Coronaviridae virus is the SARS virus and the illnesse due to the virus is SARS. Additional embodiments of this method respectively include the method as originally described and the method directed to SARS as described in the preceding sentence, wherein the nucleoside compound of Formula I employed in the pharmaceutical composition is a compound embraced by one of the embodiments, or an aspect or feature thereof, as described above. In all of these embodiments, the compound may optionally be used in the form of a pharmaceutically acceptable salt. Still further embodiments of this method include the method as originally described or as described in any of the preceding embodiments, wherein the pharmaceutical composition containing the compound of Formula I, or a pharmaceutically acceptable salt thereof, is administered in combination with one or more other agents active against against a Coronaviridae virus or against an illness due to a Coronaviridae virus, such as interferon, ribavirin, levovirin, and viramidine.

The present invention also includes a pharmaceutical composition comprising a compound of Formula I and a pharmaceutically acceptable carrier for: (i) use in, (ii) use as a medicament for, or (iii) use in the preparation of a medicament for: (a) inhibiting replication of a Coronaviridae virus (e.g., the SARS virus), (b) for treating or prophylaxis of an infection by a Coronaviridae virus, or (c) for treating or prophylaxis of an illness (e.g., SARS) due to a Coronaviridae virus. In these uses, the pharmaceutical compositions containing the compound of Formula I can optionally be employed in combination with one or more agents active against a Coronaviridae virus or against an illness due to a Coronaviridae virus, such as interferon, ribavirin, levovirin, and viramidine. Additional embodiments of the invention include any of the uses set forth in the first sentence of this paragraph, wherein the nucleoside compound of Formula I employed therein is a compound embraced by one of the embodiments, or an aspect or feature thereof, as described above. In all of these embodiments, the compound may optionally be used in the form of a pharmaceutically acceptable salt.

As used herein, the term “composition”, as in “pharmaceutical composition,” refers to a product comprising the active ingredient(s) (e.g., Compound I), the carrier and optionally other ingredients (e.g., one or more excipients), as well as any product which results, directly or indirectly, from combination, complexation or aggregation of any two or more of the components of the composition, or from dissociation of one or more of the components, or from other types of reactions or interactions of one or more of the components. Accordingly, the pharmaceutical compositions employed in the present invention include any composition which comprises the product prepared by combining a nucleoside compound of Formula I, a pharmaceutically acceptable carrier, and optionally other ingredients.

By “pharmaceutically acceptable” is meant that the ingredients of a pharmaceutical composition are compatible with each other and not deleterious to the recipient thereof.

As described above, the pharmaceutical compositions suitable for use in the present invention comprise a compound of Formula I as an active ingredient or a pharmaceutically acceptable salt thereof, and may also contain a pharmaceutically acceptable carrier and optionally other therapeutic ingredients. The compositions include compositions suitable for oral, rectal, topical, parenteral (including subcutaneous, intramuscular, and intravenous), ocular (ophthalmic), pulmonary (nasal or buccal inhalation), or nasal administration, although the most suitable route in any given case will depend on the nature and severity of the conditions being treated and on the nature of the active ingredient. They may be conveniently presented in unit dosage form and prepared by any of the methods known in the art of pharmacy.

In practical use, a compound of Formula I can be combined as the active ingredient in intimate admixture with a pharmaceutical carrier according to conventional pharmaceutical compounding techniques. The carrier can be any of a wide variety of forms depending on the form of preparation desired for administration, e.g., oral or parenteral (including intravenous). In preparing an orally administered composition, any of the usual pharmaceutical media can be employed, such as water, glycols, oils, alcohols, flavoring agents, preservatives, coloring agents and the like in the case of oral liquid preparations such as suspensions, elixirs and solutions; or carriers such as starches, sugars, microcrystalline cellulose, diluents, granulating agents, lubricants, binders, disintegrating agents and the like in the case of oral solid preparations such as powders, hard and soft capsules, and tablets. Solid oral preparations are typically preferred over liquid preparations.

Because of their ease of administration, tablets and capsules represent advantageous oral dosage forms. If desired, tablets may be coated by standard aqueous or nonaqueous techniques. Such compositions and preparations should contain at least 0.1 percent of active compound. The percentage of active compound in these compositions can, of course, be varied and can conveniently range from about 2 percent to about 60 percent of the weight of the dosage unit. The amount of active compound in such therapeutically useful compositions is such that an effective dosage will be obtained. The active compounds can also be administered intranasally as, for example, liquid drops or spray.

The tablets, pills, capsules, and the like may also contain a binder such as gum tragacanth, acacia, corn starch or gelatin; excipients such as dicalcium phosphate; a disintegrating agent such as corn starch, potato starch, alginic acid; a lubricant such as magnesium stearate; and a sweetening agent such as sucrose, lactose or saccharin. When a dosage unit form is a capsule, it may contain, in addition to materials of the above type, a liquid carrier such as a fatty oil.

Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye, and a flavoring such as cherry or orange flavor.

Compounds of Formula I may also be administered parenterally. Solutions or suspensions of these active compounds can be prepared in water suitably mixed with a surfactant such as hydroxy-propylcellulose. Dispersions can also be prepared in glycerol, liquid polyethylene glycols and mixtures thereof in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g. glycerol, propylene glycol and liquid polyethylene glycol), suitable mixtures thereof, and vegetable oils.

For the purposes of the present invention, any suitable route of administration may be employed for providing the subject (i.e., a vertebrate species, preferably a mammal, and especially a human) with an effective dosage of a nucleoside compound of Formula I. For example, oral, rectal, topical, parenteral, ocular, pulmonary, nasal, and the like may be employed. Dosage forms include tablets, troches, dispersions, suspensions, solutions, capsules, creams, ointments, aerosols, and the like. Oral administration is typically preferred.

For oral administration to humans, the dosage range is 0.01 to 1000 mg/kg body weight per day in a single dose or in divided doses. In one embodiment the dosage range is 0.1 to 100 mg/kg body weight per day in a single dose or in divided doses. In another embodiment the dosage range is 0.5 to 20 mg/kg body weight in divided doses. For oral administration, the compositions are preferably provided in the form of tablets or capsules containing 1.0 to 1000 milligrams of the active ingredient. Tablets or capsules containing 1, 5, 10, 15, 20, 25, 50, 75, 100, 150, 200, 250, 300, 400, 500, 600, 750, 800, 900, and 1000 milligrams of the active ingredient can be employed, for example.

The effective dosage of active ingredient employed may vary depending on the particular compound employed, the mode of administration, the condition being treated and the severity of the condition being treated. Such dosage may be ascertained readily by a person skilled in the art. The dosage regimen may be adjusted to provide the optimal therapeutic response.

The nucleoside compounds and derivatives thereof employed in the methods of the present invention can be prepared following synthetic methodologies well-established in the practice of nucleoside and nucleotide chemistry. Reference is made to the following text for a description of synthetic methods used in the preparation of the compounds employed in the present invention: “Chemistry of Nucleosides and Nucleotides,” L. B. Townsend, ed., Vols. 1-3, Plenum Press, 1988, the disclosure of which is incorporated by reference herein in its entirety. Preparative methods are also described in WO02/057425, the disclosure of which is incorporated by reference herein in its entirety. With respect to the preparation of thionucleoside compounds wherein the ring oxygen is replaced with a sulfur, reference is made to the following article and references cited therein for methods of preparation of thionucleosides wherein the ring oxygen is replaced with a sulfur: L. Bellon, et al., “4′-Thio-RNA: a novel class of sugar-modified β-RNA,” ACS Symposium Series (1994), 580 (Carbohydrate Modifications in Antisense Research), pages 68-79.

The synthesis of 9-(2′-C-methyl-β-D-ribofuranosyl)purines of Formula VI is described in U.S. Pat. No. 3,480,613; FR Patent 1521076; and R. E. Harry-O'kuru et al., J. Org. Chem. 1997, 62: 1754-1759, the disclosures of which are incorporated herein by reference in their entireties.

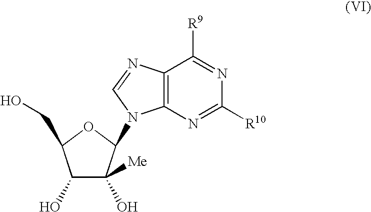

Methods for the synthesis of 9-(2′-C-branched-β-D-ribofuranosyl)pyrimidines of Formula VII are described in U.S. Pat. No. 3,480,613; GB 1209654; R. E. Harry-O'kuru et al., J. Org. Chem. 1997, 62: 1754-1759; and M. Gallo et al., Molecules 2000, 5: 727-729, the disclosures of which are incorporated herein by reference in their entireties.

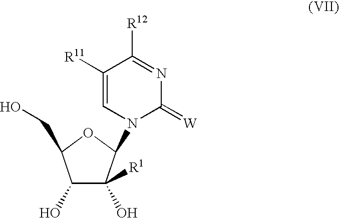

The preparation of 2′-C-alkylribofuranosyladenines is described in P. Franchetti et al., J. Med. Chem. 1998, 41: 1708-1715, the disclosure of which is herein incorporated by reference in its entirety.

A representative general method for the preparation of deazapurine compounds suitable for use in the present invention is outlined in Scheme 1 below. This scheme illustrates the synthesis of compounds embraced by Formula I which are of structural formula 1-7, wherein the furanose ring has the β-D-ribo configuration. The starting material is a 3,5-bis-O-protected alkyl furanoside, such as methyl furanoside, of structural formula 1-1. The C-2 hydroxy group is then oxidized with a suitable oxidizing agent, such as a chromium trioxide or chromate reagent, Dess-Martin periodinane, or by Swern oxidation, to afford a C-2 ketone of structural formula 1-2. Addition of a Grignard reagent, such as an alkyl, alkenyl, or alkynyl magnesium halide (for example, MeMgBr, EtMgBr, vinylMgBr, allylMgBr, and ethynylMgBr) or an alkyl, alkenyl, or alkynyl lithium, such as MeLi, across the carbonyl double bond of 1-2 in a suitable organic solvent, such as tetrahydrofuran, diethyl ether, and the like, affords the C-2 tertiary alcohol of structural formula 1-3. A good leaving group (such as Cl, Br, and I) is next introduced at the C-1 (anomeric) position of the furanose sugar derivative by treatment of the furanoside of formula 1-3 with a hydrogen halide in a suitable organic solvent, such as hydrogen bromide in acetic acid, to afford the intermediate furanosyl halide 1-4. A C-1 sulfonate, such methanesulfonate (MeSO₂O—), trifluoromethane-sulfonate (CF₃SO₂O—), or p-toluenesulfonate (—OTs), may also serve as a useful leaving group in the subsequent reaction to generate the glycosidic (nucleosidic) linkage. The nucleosidic linkage is constructed by treatment of the intermediate of structural formula 1-4 with the metal salt (such as lithium, sodium, or potassium) of an appropriately substituted 1H-pyrrolo[2,3-d]pyrimidine 1-5, such as an appropriately substituted 4-halo-1H-pyrrolo[2,3-d]pyrimidine, which can be generated in situ by treatment with an alkali hydride (such as sodium hydride), an alkali hydroxide (such as potassium hydroxide), an alkali carbonate (such as potassium carbonate), or an alkali hexamethyldisilazide (such as NaHMDS) in a suitable anhydrous organic solvent, such as acetonitrile, tetrahydrofuran, 1-methyl-2-pyrrolidinone, or N,N-dimethylformamide (DMF). The displacement reaction can be catalyzed by using a phase-transfer catalyst, such as TDA-1 or triethylbenzyl-ammonium chloride, in a two-phase system (solid-liquid or liquid-liquid). The optional protecting groups in the protected nucleoside of structural formula 1-6 are then cleaved following established deprotection methodologies, such as those described in T. W. Greene and P. G. M. Wuts, “Protective Groups in Organic Synthesis,” 3^{rd} ed., John Wiley & Sons, 1999. Optional introduction of an amino group at the 4-position of the pyrrolo[2,3-d]pyrimidine nucleus is effected by treatment of the 4-halo intermediate 1-6 with the appropriate amine, such as alcoholic ammonia or liquid ammonia, to generate a primary amine at the C-4 position (—NH₂), an alkylamine to generate a secondary amine (—NHR), or a dialkylamine to generate a tertiary amine (—NRR′). A 7H-pyrrolo[2,3-d]pyrimidin-4(3H)one compound may be derived by hydrolysis of 1-6 with aqueous base, such as aqueous sodium hydroxide. Alcoholysis (such as methanolysis) of 1-6 affords a C-4 alkoxide (—OR), whereas treatment with an alkyl mercaptide affords a C-4 alkylthio (—SR) derivative. Subsequent chemical manipulations well-known to practitioners of ordinary skill in the art of organic/medicinal chemistry may be required to attain the desired compounds of the present invention.

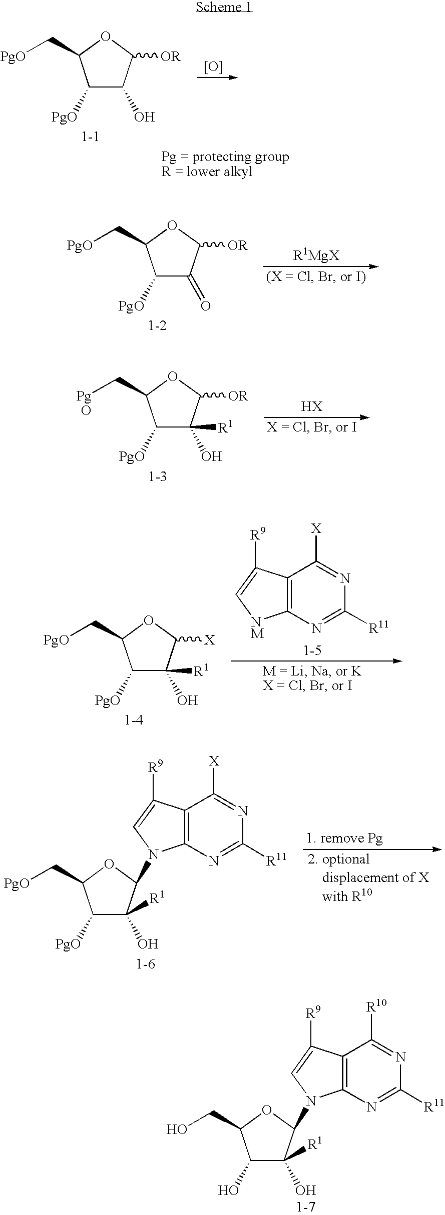

The examples below provide citations to literature publications, which contain details for the preparation of final compounds or intermediates employed in the preparation of final compounds of the present invention. The nucleoside compounds of the present invention were prepared according to procedures detailed in the following examples. The examples are not intended to be limitations on the scope of the instant invention in any way, and they should not be so construed. Those skilled in the art of nucleoside and nucleotide synthesis will readily appreciate that known variations of the conditions and processes of the following preparative procedures can be used to prepare these and other compounds of the present invention. All temperatures are degrees Celsius unless otherwise noted.

### EXAMPLE 1

4-Amino-7-(2-C-methyl-β-D-ribofuranosyl)-7H-pyrrolo[2,3-d]pyrimidine

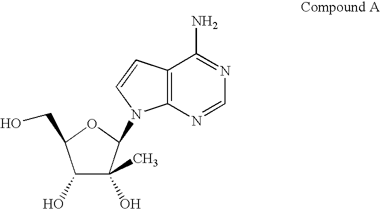

Step A: 3,5-Bis-O-(2,4-dichlorophenylmethyl)-1-O-methyl-α-D-ribofuranose

A mixture of 2-O-acetyl-3,5-bis-O-(2,4-dichlorophenylmethyl)-1-O-methyl-α-D-ribofuranose [for preparation, see: Helv. Chim. Acta 78: 486 (1995)] (52.4 g, 0.10 mol) in methanolic K₂CO₃ (500 mL, saturated at room temperature) was stirred at room temperature for 45 min and then concentrated under reduced pressure. The oily residue was suspended in CH₂Cl₂ (500 mL), washed with water (300 mL+5×200 mL) and brine (200 mL), dried (Na₂SO₄), filtered, and concentrated to give the title compound (49.0 g) as colorless oil, which was used without further purification in Step B below.

¹H NMR (DMSO-d₆): δ 3.28 (s, 3H, OCH₃), 3.53 (d, 2H, J_{5.4}=4.5 Hz, H-5a, H-5b), 3.72(dd, 1H, J_{3.4}=3.6 Hz, J_{3.2}=6.6 Hz, H-3), 3.99 (ddd, 1H, J_{2.1}=4.5 Hz, J_{2OH-2}=9.6 Hz, H-2), 4.07 (m, 1H, H-4), 4.50 (s, 2H, CH₂Ph), 4.52, 4.60 (2d, 2H, J_{gem}=13.6 Hz, CH₂Ph), 4.54 (d, 1H, OH-2), 4.75 (d, 1H, H-1), 7.32-7.45, 7.52-7.57 (2m, 10H, 2Ph).

¹³C NMR (DMSO-d₆): δ 55.40, 69.05, 69.74, 71.29, 72.02, 78.41, 81.45, 103.44, 127.83, 127.95, 129.05, 129.28, 131.27, 131.30, 133.91, 133.55, 133.67, 135.45, 135.92.

Step B: 3,5-Bis-O-(2,4-dichlorophenylmethyl)-1-O-methyl-α-D-erythro-pentofuranos-2-ulose

To an ice-cold suspension of Dess-Martin periodinane (50.0 g, 118 mmol) in anhydrous CH₂Cl₂ (350 mL) under argon (Ar) was added a solution of the compound from Step A (36.2 g, 75 mmol) in anhydrous CH₂Cl₂ (200 mL) dropwise over 0.5 h. The reaction mixture was stirred at 0° C. for 0.5 h and then at room temperature for 3 days. The mixture was diluted with anhydrous Et₂O (600 mL) and poured into an ice-cold mixture of Na₂S₂O₃.5H₂O (180 g) in saturated aqueous NaHCO₃ (1400 mL). The layers were separated, and the organic layer was washed with saturated aqueous NaHCO₃ (600 mL), water (800 mL) and brine (600 mL), dried (MgSO₄), filtered and evaporated to give the title compound (34.2 g) as a colorless oil, which was used without further purification in Step C below.

¹H NMR (CDCl₃): δ 3.50 (s, 3H, OCH₃), 3.79 (dd, 1H, J_{5a,5b}=11.3 Hz, J_{5a, 4}=3.5 Hz, H-5a), 3.94 (dd, 1H, J_{5b,4}=2.3 Hz, H-5b), 4.20 (dd, 1H, J_{3.1}=1.3 Hz, J_{3.4}=8.4 Hz, H-3), 4.37 (ddd, 1H, H-4), 4.58, 4.69 (2d, 2H, J_{gem}=13.0 Hz, CH₂Ph), 4.87 (d, 1H, H-1), 4.78, 5.03 (2d, 2H, J_{gem}=12.5 Hz, CH₂Ph), 7.19-7.26, 7.31-7.42 (2m, 10H, 2Ph).

¹³C NMR (DMSO-d₆): δ 55.72, 69.41, 69.81, 69.98, 77.49, 78.00, 98.54, 127.99, 128.06, 129.33, 129.38, 131.36, 131.72, 133.61, 133.63, 133.85, 133.97, 134.72, 135.32, 208.21.

Step C: 3,5-Bis-O-(2,4-dichlorophenyl methyl)-2-C-methyl-1-O-methyl-α-D-ribofuranose

To a solution of MeMgBr in anhydrous Et₂O (0.48 M, 300 mL) at −55° C. was added dropwise a solution of the compound from Step B (17.40 g, 36.2 mmol) in anhydrous Et₂O (125 mL). The reaction mixture was allowed to warm to −30° C. and stirred for 7 h at −30° C. to −15° C., then poured into ice-cold water (500 mL) and the mixture vigorously stirred at room temperature for 0.5 h. The mixture was filtered through a Celite pad (10×5 cm) which was thoroughly washed with Et₂O. The organic layer was dried (MgSO₄), filtered and concentrated. The residue was dissolved in hexanes (˜30 mL), applied onto a silica gel column (10×7 cm, prepacked in hexanes) and eluted with hexanes and hexanes/EtOAc (9/1) to give the title compound (16.7 g) as a colorless syrup.

¹H NMR (CDCl₃): δ 1.36 (d, 3H, J_{Me.OH}=0.9 Hz, 2C-Me), 3.33 (q, 1H, OH), 3.41 (d, 1H, J_{3,4}=3.3 Hz), 3.46 (s, 3H, OCH₃), 3.66 (d, 2H, J_{5.4}=3.7 Hz, H-5a, H-5b), 4.18 (apparent q, 1H, H-4), 4.52 (s, 1H, H-1), 4.60 (s,2H. CH₂Ph), 4.63, 4.81(2d, 2H, J_{gem}=13.2 Hz, CH₂Ph), 7.19-7.26,7,34-7.43 (2m, 10H, 2Ph).

¹³C NMR (CDCl₃): δ 24.88, 55.45, 69.95, 70.24, 70.88, 77.06, 82.18, 83.01, 107.63, 127.32, 129.36, 130.01, 130.32, 133.68, 133.78, 134.13, 134.18, 134.45, 134.58.

Step D: 4-Chloro-7-[3,5-bis-O-(2,4-dichlorophenylmethyl)-2-C-methyl-β-D-ribofuranosyl]-7H-pyrrolo[2,3-d]pyrimidine

To a solution of the compound from Step C (9.42 g, 19 mmol) in anhydrous dichloromethane (285 mL) at 0° C. was added HBr (5.7 M in acetic acid, 20 mL, 114 mmol) dropwise. The resulting solution was stirred at 0° C. for 1 h and then at room temperature for 3 h, evaporated in vacuo and co-evaporated with anhydrous toluene (3×40 mL). The oily residue was dissolved in anhydrous acetonitrile (50 mL) and added to a solution of the sodium salt of 4-chloro-1H-pyrrolo[2,3-d]pyrimidine in acetonitrile [generated in situ from 4-chloro-1H-pyrrolo[2,3-d]pyrimidine [for preparation, see J. Chem. Soc., 131 (1960)] (8.76 g, 57 mmol) in anhydrous acetonitrile (1000 mL), and NaH (60% in mineral oil, 2.28 g, 57 mmol), after 4 h of vigorous stirring at room temperature]. The combined mixture was stirred at room temperature for 24 h, and then evaporated to dryness. The residue was suspended in water (250 mL) and extracted with EtOAc (2×500 mL). The combined extracts were washed with brine (300 mL, dried over Na₂SO₄, filtered and evaporated. The crude product was purified on a silica gel column (10 cm×10 cm) using ethyl acetate/hexane (1:3 and 1:2) as the eluent. Fractions containing the product were combined and evaporated in vacuo to give the desired product (5.05 g) as a colorless foam.

¹H NMR (CDCl₃): δ 0.93 (s, 3H, CH₃), 3.09 (s, 1H, OH), 3.78 (dd, 1H, J_{5′,5″}=10.9 Hz, J_{5,4}=2.5 Hz, H—S “), 3.99 (dd, 1H, J_{5″,4}=2.2 Hz, H—S”), 4.23-4.34 (m, 2H, H-3′, H-4′), 4.63, 4.70 (2d, 2H, J_{gem}=12.7 Hz, CH₂Ph), 4.71, 4.80 (2d, 2H, J_{gem}=12.1 Hz,CH₂Ph), 6.54 (d, 1H, J_{5,6}=3.8 Hz, H-5), 7.23-7.44 (m, 10H, 2Ph).

¹³C NMR (CDCl₃): δ 21.31, 69.10, 70.41, 70.77, 79.56, 80.41, 81.05, 91.11, 100.57, 118.21, 127.04, 127.46, 127.57, 129.73, 129.77, 130.57, 130.99, 133.51, 133.99, 134.33, 134.38, 134.74, 135.21, 151.07, 151.15, 152.47.

Step E: 4-Chloro-7-(2-C-methyl-β-D-ribofuranosyl)-7H-pyrrolo[2,3-d]pyrimidine

To a solution of the compound from Step D (5.42 g, 8.8 mmol) in dichloromethane (175 mL) at −78° C. was added boron trichloride (1 M in dichloromethane, 88 mL, 88 mmol) dropwise. The mixture was stirred at −78° C. for 2.5 h, then at −30° C. to −20° C. for 3 h. The reaction was quenched by addition of methanol/dichloromethane (1:1) (90 mL) and the resulting mixture stirred at −15° C. for 30 min, then neutralized with aqueous ammonia at 0° C. and stirred at room temperature for 15 min. The solid was filtered and washed with CH₂Cl₂/MeOH (1/1, 250 mL). The combined filtrate was evaporated, and the residue was purified by flash chromatography over silica gel using CH₂Cl₂ and CH₂Cl₂:MeOH (99:1, 98:2, 95:5 and 90:10) gradient as the eluent to furnish desired compound (1.73 g) as a colorless foam, which turned into an amorphous solid after treatment with MeCN.

¹H NMR (DMSO-d₆): δ 0.64 (s, 3H, CH₃), 3.61-3.71 (m, 1H, H-5′), 3.79-3.88 (m, 1H, H-5″), 3.89-4.01 (m, 2H, H-3′, H-4′), 5.15-5.23 (m, 3H, 2′-OH, 3′-OH, 5′-OH), 6.24 (s, 1H, H-1′), 6.72 (d, 1H, J_{5,6}=3.8 Hz, H-5), 8.13 (d, 1H, H-6), 8.65 (s, 1H, H-2).

¹³C NMR (DMSO-d₆): δ 20.20, 59.95, 72.29, 79.37, 83.16, 91.53, 100.17, 117.63, 128.86, 151.13, 151.19, 151.45.

Step F: 4-Amino-7-(2-C-methyl-β-D-ribofuranosyl)-7H-pyrrolo[2,3-d]pyrimidine To the compound from Step E (1.54 g, 5.1 mmol) was added methanolic ammonia (saturated at 0° C.; 150 mL). The mixture was heated in a stainless steel autoclave at 85° C. for 14 h, then cooled and evaporated in vacuo. The crude mixture was purified on a silica gel column with CH₂Cl₂/MeOH (9/1) as eluent to give the title compound as a colorless foam (0.8 g), which separated as an amorphous solid after treatment with MeCN. The amorphous solid was recrystallized from methanol/acetonitrile; m.p. 222° C.

¹H NMR (DMSO-d₆): δ 0.62 (s, 3H, CH₃), 3.57-3.67 (m, 1H, H-5′), 3.75-3.97 (m, 3H, H-5″, H-4′, H-3′), 5.00 (s, 1H, 2′-OH), 5.04 (d, 1H, J₃′OH,3′=6.8 Hz, 3′-OH), 5.06 (t, 1H, J_{5′OH,5′.5″}=5.1 Hz, 5′-OH), 6.11 (s, 11H, H—I “), 6.54 (d, 11H, J_{5,6}=3.6 Hz, H-5), 6.97 (br s, 2H, NH₂), 7.44 (d, 1H, H-6), 8.02 (s, 1H, H-2).

¹³C NMR (DMSO-d₆): δ 20.26, 60.42, 72.72, 79.30, 82.75, 91.20, 100.13, 103.08, 121.96, 150.37, 152.33, 158.15. LC-MS: Found: 279.10 (M−H⁺); calc. for C₁₂H₁₆N₄O₄+H⁺: 279.11.

### EXAMPLE 2

### Biological Assays

The assays employed to measure activity against coronavirus are described below (wherein CPE=cytopathic effect; FBS=fetal bovine serum; MEM=minimal essential medium; MOI=multiplicity of infection; and NR=neutral red):

a. Viruses and Cells

Human coronavirus OC43 was obtained from the American Type Culture Collection (ATCC, Manassas, Va., USA). This virus was originally isolated from the tracheal washings of a male with cold-like symptoms.

B-SC-1 cells propagated from adult African green kidney cells were grown in MEM supplemented with 0.1% NaHCO₃ and 10% FBS. When performing antiviral assays, serum was reduced to 2% and 50 μg/mL gentamicin was added to the medium.

b. Determination of In Vitro Antiviral Activity of Compounds Against a Coronavirus

(Cytopathic Effect Inhibition Assay)

Each compound was diluted in MEM using a four log dilution series (i.e., 100, 10, 1.0, 0.1 μg/ml) and 100 μL added to the appropriate wells of a 96 well cell culture plate with confluent cells (see plate format below). The coronavirus was diluted in MEM 2% FBS to an MOI=0.001 in a volume of 100 μL and added immediately to plate (usually within 5 minutes of drug addition). After infection, the control and compound-treated plates were incubated at 37° C. until the untreated control wells were observed to have 75-100% cytopathic effect (6 days). The plates were then scored for virus cytopathic effect at each dilution of compound tested. The 50% effective concentration (EC₅₀) was calculated by regression analysis using the means of the CPE ratings at each concentration of compound.

The compound prepared in Example 1,2′-C-methylcytidine (the preparation of which is disclosed in WO02/057425 (see example 122)), and 2′-C-methyladenosine (the preparation of which is disclosed in P. Franchetti et al., J. Med. Chem. 1998, 41: 1708-1715) were tested in this assay and exhibited EC₅₀ values of less than 100 micromolar.

c. Determination of In Vitro Antiviral Activity of Compounds Against a Coronavirus

(Neutral Red Uptake Assay)

After performing the CPE inhibition assay above, an additional cytopathic detection method was used, in which medium was removed from each well of a plate scored for CPE from a CPE inhibition assay, 0.034% NR was added to each well of the plate and the plate incubated for 2 hr at 37° C. in the dark. The NR solution was then removed from the wells. After rinsing and aspirating to dryness, the remaining dye was extracted for 30 min at room temperature in the dark from the cells using absolute ethanol buffered with Sörenson citrate buffer. After scoring each plate for cytopathic effects, each plate was then incubated with neutral red (NR) for 2 h at 37° C. Absorbances at 540 nm and 450 nm were read with a microplate reader (Bio-Tek EL 1309; Bio-Tek Instruments, Inc., Winooski, Vt.). Absorbance values were expressed as percentages of untreated controls and EC₅₀ and IC₅₀ values were calculated by regression analysis.

The compound prepared in Example 1,2′-C-methylcytidine, and 2′-C-methyladenosine were tested in this assay and exhibited EC₅₀ values of 10 micromolar or less.

### EXAMPLE 3

### Counterscreens

Nucleoside compounds suitable for use in the present invention were also evaluated for cellular toxicity and antiviral specificity in several counterscreens.

a. DNA Polymerase

The ability of the nucleoside derivatives of the present invention to inhibit human DNA polymerases was measured in the following assays:

al. Inhibition of Human DNA Polymerases Alpha and Beta: Reaction Conditions:

50 μL reaction volume

Reaction Buffer Components:

0.20 mM Tris-HCl, pH 7.5

200 μg/mL bovine serum albumin

100 mM KCl

2 mM β-mercaptoethanol

10 mM MgCl₂

1.6 μM dATP, dGTP, dCTP, dTTP

α-³³P-dATP

Enzyme and Template:

0.05 mg/mL gapped fish sperm DNA template

U/μL DNA polymerase α or β

Preparation of Gapped Fish Sperm DNA Template:

Add 5 μL 1M MgCl₂ to 500 μL activated fish sperm DNA (USB 70076);

Warm to 37° C. and add 30 μL of 65 U/μL of exonuclease III (GibcoBRL 18013-011);

Incubate 5 min at 37° C.;

Terminate reaction by heating to 65° C. for 10 min;

Load 50-100 μL aliquots onto Bio-spin 6 chromatography columns (Bio-Rad 732-6002) equilibrated with 20 mM Tris-HCl, pH 7.5;

Elute by centrifugation at 1,000×g for 4 min;

Pool eluate and measure absorbance at 260 nm to determine concentration.

The DNA template was diluted into an appropriate volume of 20 mM Tris-HCl, pH 7.5 and the enzyme was diluted into an appropriate volume of 20 mM Tris-HCl, containing 2 mM β-mercaptoethanol, and 100 mM KCl. Template and enzyme were pipetted into microcentrifuge tubes or a 96 well plate. Blank reactions excluding enzyme and control reactions excluding test compound were also prepared using enzyme dilution buffer and test compound solvent, respectively. The reaction was initiated with reaction buffer with components as listed above. The reaction was incubated for 1 h at 37° C. The reaction was quenched by the addition of 20 μL 0.5M EDTA. 50 μL of the quenched reaction was spotted onto Whatman DE81 filter disks and air dried. The filter disks were repeatedly washed with 150 mL 0.3M ammonium formate, pH 8 until 1 mL of wash is <100 cpm. The disks were washed twice with 150 mL absolute ethanol and once with 150 mL anhydrous ether, dried and counted in 5 mL scintillation fluid.

The percentage of inhibition was calculated according to the following equation: % inhibition=[1−(cpm in test reaction−cpm in blank)/(cpm in control reaction cpm in blank)]×100.

a.2. Inhibition of Human DNA Polymerase Gamma:

The potential for inhibition of human DNA polymerase gamma was measured in reactions that included 0.5 ng/μL enzyme; 10 μM dATP, dGTP, dCTP, and TTP; 2 μCi/reaction [α-³³P]-dATP, and 0.4 μg/μL activated fish sperm DNA (purchased from US Biochemical) in a buffer containing 20 mM Tris pH8, 2 mM β-mercaptoethanol, 50 mM KCl, 10 mM MgCl2, and 0.1 μg/μL BSA. Reactions were allowed to proceed for 1 h at 37° C. and were quenched by addition of 0.5 M EDTA to a final concentration of 142 mM. Product formation was quantified by anion exchange filter binding and scintillation counting. Compounds were tested at up to 50 μM.

The percentage of inhibition was calculated according to the following equation: % inhibition=[1-(cpm in test reaction−cpm in blank)/(cpm in control reaction−cpm in blank)]×100.

b. HIV Inhibition

The ability of the nucleoside compounds to inhibit HIV infectivity and HIV spread was measured in the following assays.

b.1. HIV Infectivity Assay

Assays were performed with a variant of HeLa Magi cells expressing both CXCR4 and CCR5 selected for low background β-galactosidase (β-gal) expression. Cells were infected for 48 h, and β-gal production from the integrated HIV-1 LTR promoter was quantified with a chemiluminescent substrate (Galactolight Plus, Tropix, Bedford, Mass.). Inhibitors were titrated (in duplicate) in twofold serial dilutions starting at 100 μM; percent inhibition at each concentration was calculated in relation to the control infection.

b.2. Inhibition of HIV Spread

The ability of the compounds of the present invention to inhibit the spread of the human immunedeficiency virus (HIV) was measured by the method described in U.S. Pat. No. 5,413,999 and J. P. Vacca, et al., Proc. Natl. Acad. Sci. 1994, 91: 4096-4100, which are incorporated by reference herein in their entirety.

c. Cytotoxicity

The nucleoside compounds for use in the present invention were also screened for cytotoxicity against cultured hepatoma (HuH-7) cells containing a subgenomic HCV Replicon in an MTS cell-based assay as described in the assay below. The HuH-7 cell line is described in H. Nakabayashi, et al., Cancer Res. 1982, 42: 3858.

c.1. Assay:

Cells were plated at 15-20,000 cells/well in 100 μL of appropriate media and incubated 18 h at 37° C., 5% CO₂. 100 μL of compound diluted in complete media was added to the cells for a final of 1% DMSO concentration. The plates were incubated at 37° C. and 5% CO₂ for 24 h. After the incubation period, 40 μL of CellTiter 96 Aqueous One Solution Cell Proliferation Assay reagent (MTS) (Promega) was added to each well, and the plates were incubated at 37° C. and 5% CO₂ for 1 h. The plates were agitated to mix well and absorbance at 490 nm was read using a plate reader. Metabolically active cells reduce MTS to formazan. Formazan absorbs at 490 nm. The absorbance at 490 nm in the presence of compound was compared to absorbance in cells without any compound added.

Reference: Cory, A. H. et al., “Use of an aqueous soluble tetrazolium/formazan assay for cell growth assays in culture,” Cancer Commun. 1991, 3: 207.

### EXAMPLE 4

Oral Pharmaceutical Formulation

As a specific embodiment of an orally administrable composition suitable for use in the present invention, 50 mg of the compound of Example 1 is formulated with sufficient finely divided lactose to provide a total amount of 580 to 590 mg to fill a size O hard gelatin capsule.

While the foregoing specification teaches the principles of the present invention, with examples provided for the purpose of illustration, the practice of the invention encompasses all of the usual variations, adaptations and/or modifications that come within the scope of the following claims.

1. A method for inhibiting replication of a Coronaviridae virus, for treating or prophylaxis of an infection by a Coronaviridae virus, or for treating or prophylaxis of an illness due to a Coronaviridae virus in a subject in need thereof, which comprises administering to the subject an inhibition effective amount or a therapeutically or prophylactically effective amount of a compound of Formula I, or a pharmaceutically acceptable salt thereof: 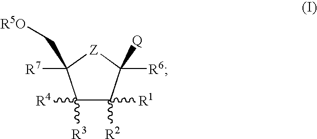
wherein
Z is O or S;
R₁ is H, OH, C₂₋₄ alkenyl, C₂₋₄ alkynyl, C₁₋₄ alkyl-O—, C₁₋₄ alkyl, C₁₋₄ haloalkyl, or C₁₋₄ alkyl substituted with from 1 to 4 substituents each of which is independently OH, amino, C₁₋₄ alkyl-O—, or C₁₋₄ alkyl-S—;
R² is H, OH, amino, halogen, C₁₋₄ alkyl-CH(NH₂)-carbonyloxy, C₁₋₁₆ alkylcarbonyloxy, mercapto, C₁₋₄ alkyl-O—, C₁₋₄ alkyl, C₁₋₄ haloalkyl, or C₁₋₄ alkyl substituted with from 1 to 4 substituents each of which is independently OH, amino, C₁₋₄ alkyl-O—, or C₁₋₄ alkyl-S—;
R³ is H, OH, cyano, azido, halogen, C₁₋₄ alkyl-CH(NH₂)-carbonyloxy, C₁₋₁₆ alkylcarbonyloxy, C₂₋₁₈ alkenylcarbonyloxy, C₄₋₁₈ polyalkenylcarbonyloxy, C₁₋₁₀ alkyloxycarbonyloxy, C₃₋₆ cycloalkylcarbonyloxy, C₃₋₆ cycloalkyloxycarbonyloxy, mercapto, amino, C₁₋₄ alkyl-O—, C₂₋₄ alkenyl, C₂₋₄ alkynyl, C₁₋₄ alkyl, C₁₋₄ haloalkyl, or C₁₋₄ alkyl substituted with from 1 to 4 substituents each of which is independently OH, amino, C₁₋₄ alkyl-O—, or C₁₋₄ alkyl-S—;
R⁴ is H, OH, cyano, azido, halogen, C₁₋₁₆ alkylcarbonyloxy, C₂₋₁₈ alkenylcarbonyloxy, C₄₋₁₈ polyalkenylcarbonyloxy, C₁₋₁₀ alkyloxycarbonyloxy, C₃₋₆ cycloalkylcarbonyloxy,
C₃₋₆ cycloalkyloxycarbonyloxy, mercapto, amino, C₁₋₄ alkyl-O—, C₂₋₄ alkenyl, C₂₋₄ alkynyl, C₁₋₄ alkyl, C₁₋₄ haloalkyl, or C₁₋₄ alkyl substituted with from 1 to 4 substituents each of which is independently OH, amino, C₁₋₄ alkyl-O—, or C₁₋₄ alkyl-S—;
R⁵ is H, C₁₋₄ alkyl-CH(NH₂)-carbonyloxy, C₁₋₁₆ alkylcarbonyl, C₂₋₁₈ alkenylcarbonyl, C₄₋₁₈ polyalkenylcarbonyloxy, C₁₋₁₀alkyloxycarbonyl, C₃₋₆ cycloalkylcarbonyl, C₃₋₆ cycloalkyloxycarbonyl, P₃O₉H₄, P₂O₆H₃, or P(O)R^{u}R^{v};
R⁶ is H, methyl, hydroxymethyl, or fluoromethyl;
R⁷ is H, methyl, hydroxymethyl, fluoromethyl, aminomethyl, azido, or cyano;
Q is: 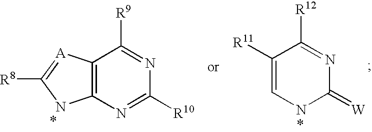
wherein
* denotes the point of attachment of Q to the C-1carbon of the furanose ring;
A is N or C—R^{w};
W is O or S;
R⁸ is H, C₁₋₄ alkyl, C₂₋₄ alkynyl, halogen, cyano, carboxy, C₁₋₄ alkyloxycarbonyl, azido, amino, C₁₋₄ alkylamino, di(C₁₄ alkyl)amino, OH, C₁₋₆ alkyl-O—, C₁₋₆ alkyl-S—, C₁₋₆ alkyl-SO₂—, aminomethyl, or (C₁₋₄ alkyl)₁₋₂aminomethyl;
R⁹ and R¹² are each independently H, OH, mercapto, halogen, C₁₋₄ alkyl-O—, C₁₋₄ alkyl-S—, C₁₋₈ alkylcarbonyloxy, C₃₋₆ cycloalkylcarbonyloxy, C₁₋₈ alkyloxycarbonyloxy, C₃₋₆ cycloalkyloxycarbonyloxy, —OCH₂CH₂SC(═O)C₁₋₄ alkyl, —OCH₂OC(═O)C₁₋₄ alkyl, —OCH(C₁₋₄ alkyl)OC(═O)C₁₋₄ alkyl, amino, C₁₋₄ alkylamino, di(C₁₋₄ alkyl)amino, C₃₋₆ cycloalkylamino, di(C₃₋₆ cycloalkyl)amino, or an amino acyl residue of formula: 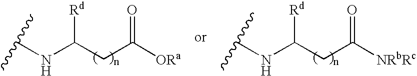
wherein n is an integer equal to zero, 1, 2, 3 or 4;
R₁₀ is H, OH, mercapto, halogen, C₁₋₄ alkyl-O—, C₁₋₄ alkyl-S—, amino, C₁₋₄ alkylamino, di(Cl 4 alkyl)amino, C₃₋₆ cycloalkylamino, di(C₃₋₆ cycloalkyl)amino, phenyl-C₁₋₂ alkylamino, C₁₋₄ alkyl-C(═O)NH—, C₁₋₈ alkylcarbonyloxy, or —OCH(C₁₋₄ alkyl)OC(═O)C₁₄ alkyl;
R¹¹ is H, C₁₋₆ alkyl, C₂₋₆ alkenyl, C₂₋₆ alkynyl, C₁₋₄ alkylamino, CF₃, or halogen;
R^{a}, R^{b}, and R^{c} are each independently H or C₁₋₆ alkyl;
R^{d} is H, C₁₋₄ alkyl, phenyl-C₁₋₂ alkyl, or phenyl;
R^{u} and R^{v} are each independently OH, —OCH₂CH₂SC(═O)C₁₋₄ alkyl, —OCH₂OC(═O)OC₁₋₄ alkyl, —NHCHMeCO₂Me, —OCH(C₁₋₄ alkyl)OC(═O)C₁₋₄ alkyl, 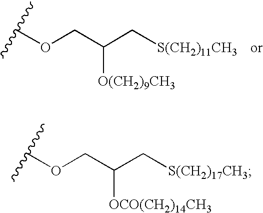
R^{w} is H, cyano, nitro, NHC(═O)NH₂, C(═O)NR^{x}R^{x}, CSNR^{x}R^{x}, C(═O)OR^{x}, C(═NH)NH₂, OH, C₁₋₃ alkoxy, amino, C₁₋₄ alkylamino, di(C₁₋₄ alkyl)amino, halogen, C₁₋₃ alkyl, or C₁₋₃ alkyl substituted with from one to three groups independently selected from halogen, amino, OH, carboxy, and C₁₋₃ alkyl-O—; and
each Rx is independently H or C₁₋₆ alkyl.

2. The method according to claim 1, wherein
Z is O;
R¹ is H, OH, C₁₋₃ alkyl-O—, C₁₋₃ alkyl, C₁₋₃ haloalkyl, or C₁₋₃ alkyl mono-substituted with OH, amino, C₁₋₄ alkyl-O— or C₁₋₄ alkyl-S—;
R² is H, OH, amino, fluoro, C₁₋₄ alkyl-CH(NH₂)-carbonyloxy, C₁₋₁₆ alkylcarbonyloxy, mercapto, C₁₋₃ alkyl-O—, C₁₋₃ alkyl, or C₁₋₃ haloalkyl;
R³ is H, OH, halogen, C₁₋₄ alkyl-CH(NH₂)-carbonyloxy, C₁₋₁₆ alkylcarbonyloxy, amino, C₁₋₃ alkyl-O—, C₁₋₃ alkyl, or C₁₋₃ haloalkyl;
R⁴ is H, OH, halogen, C₁₋₁₆ alkylcarbonyloxy, amino, C₁₋₃ alkyl-O—, C₁₋₃ alkyl, or C₁₋₃ haloalkyl;
R⁵ is H, C₁₋₄ alkyl-CH(NH₂)-carbonyl, C₁₋₁₆ alkylcarbonyl, P₃O₉H₄, P₂O₆H₃, or PO₃H₂; and
R⁶ and R⁷ are both H.

3. The method according to claim 2, wherein
R¹ is H, OH, methyl, methoxy, fluoromethyl, hydroxymethyl, difluoromethyl, trifluoromethyl, or aminomethyl;
R² is H, OH, fluoro, (CH₃)₂CHCH(NH₂)-carbonyloxy, C₁₋₁₆ alkylcarbonyloxy, or methoxy;
R³ is H, OH, fluoro, (CH₃)₂CHCH(NH₂)-carbonyloxy, C₁₋₁₆ alkylcarbonyloxy, amino, or methoxy;
R⁴ is H; and
R⁵ is H, (CH₃)₂CHCH(NH₂)-carbonyl, C₁₋₁₆ alkylcarbonyl, or P₃O₉H₄.

4. The method according to claim 3, wherein
R¹ is methyl; and
R² and R³ are both OH.

5. The method according to claim 1, wherein
Q is: 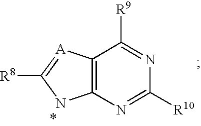
A is N or C-R^{w};
R⁸ is H, C₁₋₃ alkyl, halogen, azido, amino, C₁₋₄ alkylamino, or C₁₋₃ alkyl-O—;
R⁹ and R¹⁰ are each independently H, OH, halogen, amino, C₁₋₄ alkylamino, di(C₁₋₄ alkyl)amino, or C₃₋₆ cycloalkylamino; and
R_{w} is hydrogen, cyano, methyl, halogen, or C(═O)NH₂.

6. The method according to claim 5, wherein the compound is a compound of Formula II, or a pharmaceutically acceptable salt thereof:

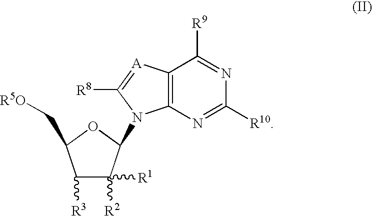

7. The method according to claim 6, wherein
R¹ is H, OH, methyl, methoxy, fluoromethyl, hydroxymethyl, difluoromethyl, trifluoromethyl, or aminomethyl;
R² is H, OH, fluoro, C₁₋₁₆ alkylcarbonyloxy, or methoxy;
R³ is H, OH, fluoro, (CH₃)₂CHCH(NH₂)-carbonyloxy, C-₁₋₁₆ alkylcarbonyloxy, amino, or methoxy; and
R⁵ is H, (CH₃)₂CHCH(NH₂)-carbonyl, C₁₋₁₆ alkylcarbonyl, or P₃O₉H₄.

8. The method according to claim 7, wherein
R¹ is methyl;
R² and R³ are both OH; and
R⁵ is H, (CH₃)₂CHCH(NH₂)-carbonyl, C₁₋₁₆ alkylcarbonyl, or P₃O₉H₄.

9. The method according to claim 8, wherein the compound is selected from the group consisting of:
4-amino-7-(2-C-methyl-β-D-ribofuranosyl)-7H-pyrrolo[2,3-d]pyrimidine;
2′-C-methyladenosine;
corresponding 5′-triphosphates thereof;
and pharmaceutically acceptable salts thereof.

10. The method according to claim 9, wherein the compound is 4-amino-7-(2-C-methyl-β-D-ribofuranosyl)-7H-pyrrolo[2,3-d]pyrimidine, or a pharmaceutically acceptable salt thereof.

11. The method according to claim 1, wherein
Q is 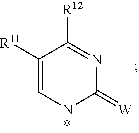
W is O;
R¹¹ is H, C₁₋₃ alkyl, C₁₋₃ alkylamino, or halogen; and
R¹² is H, OH, halogen, amino, C₁₋₄ alkylamino, di(C₁₋₄ alkyl)amino, or C₃₋₆ cycloalkylamino.

12. The method according to claim 11, wherein
Z is 0;
R¹ is H, OH, methyl, methoxy, fluoromethyl, hydroxymethyl, difluoromethyl, trifluoromethyl, or aminomethyl;
R² is H, OH, fluoro, C₁₋₁₆ alkylcarbonyloxy, or methoxy;
R³ is H, OH, fluoro, (CH₃)₂CHCH(NH₂)-carbonyloxy, C₁₋₁₆ alkylcarbonyloxy, amino, or methoxy;
R⁴ is H;
R⁵ is H, (CH₃)₂CHCH(NH₂)-carbonyl, C₁₋₁₆ alkylcarbonyl, or P₃O₉H₄; and
R⁶ and R⁷ are both H.

13. The method according to claim 12, wherein
R¹ is methyl; and
R² and R³ are both OH.

14. The method according to claim 13, wherein the compound is 2′-C-methylcytidine, or a pharmaeutically acceptable salt thereof.

15. The method according to claim 1, which is a method for inhibiting replication of a Coronaviridae virus.

16. (canceled)

17. The method according to claim 15, wherein the Coronaviridae virus is the SARS virus.

18-21. (canceled)

22. The method according to claim 1, which is a method for treating or prophylaxis of infection by a Coronaviridae virus.

23. (canceled)

24. The method according to claim 22, wherein the Coronaviridae virus is the SARS virus.

25-28. (canceled)

29. The method according to claim 1, which is a method for treating or prophylaxis of an illness due to a Coronaviridae virus.

30. The method according to claim 29, wherein the illness is SARS.

31-44. (canceled)
